(19) United States
(12) Patent Application Publication
(10) Pub. No.: US 2019/0365309 A1
(43) Pub. Date: Dec. 5, 2019

(54) ADJUSTMENT DEVICE FOR VESTIBULAR SYSTEM OF HUMAN BODY AND APPLICATION THEREFOR

(71) Applicant: Cheng-Chia LI, Taichung (TW)

(72) Inventor: Cheng-Chia LI, Taichung (TW)

(21) Appl. No.: 15/990,903

(22) Filed: May 29, 2018

(51) Int. Cl. A61B 5/00

(52) U.S. Cl. CPC A61B 5/4023; A61B 5/0476; A61B 5/6887

(57) ABSTRACT

An adjustment device for vestibular system of human body contains: a control module including a rotatable fulcrum unit, and the rotatable fulcrum unit having a drive unit which has four support portions which obliquely rotate upward and downward around the rotatable fulcrum unit in a circumferential operation path. The drive unit includes a motor, a drive wheel, a deceleration wheel, a belt, an actuation shaft, and two transmission members. Each universal connection rod has a universal output segment opposite to the universal input segment so that the universal input segment outputs power to the universal output segment, and the universal output segment is connected with the rotatable fulcrum unit. The carrier is connected with the four support portions of the drive unit.

### FIELD OF THE INVENTION

The present invention relates to an adjustment device for vestibular system of human body which has multiple support portions obliquely rotating upward and downward around a circumferential operation path.

### BACKGROUND OF THE INVENTION

Vestibular systems of two inner ears of human body detect head movement and position change by using fluid-filled tubes. The vestibular systems connect with cochlea, and vestibular apparatus contains: otolith organs in utricle and saccule so as to detect linear acceleration in the head; three semicircular canals formed in X, Y, and Z axes respectively and perpendicular to one another so as to detect angular acceleration of the head. After the stimulus signals are transmitted to the vestibular nucleus, they are sent to third, fourth, and sixth pairs of brain nerves of eyeballs and are sent by vestibular nucleus to intrinsic muscle via vestibulovpinal tract so as to be integrated.

A fatigue of eye muscle occurs after staring at the screen for a long time, and spine curve changes to cause pains of shoulder, neck, and lower back and scoliosis.

A research report published in 2012 discloses that the frog starts scoliosis after damaging its vestibular system of inner ear.

The present invention has arisen to mitigate and/or obviate the afore-described disadvantages.

### SUMMARY OF THE INVENTION

The primary objective of the present invention is to provide an adjustment device for vestibular system of human body which has multiple support portions obliquely rotating upward and downward around a circumferential operation path.

Another objective of the present invention is to provide application of the vestibular system of the human body contains: activating vestibular system of children and improving brain development; relieving pains of shoulder, neck, and lower back; stabilizing emotions and easing anxiety; activating the brain and delaying degradation of the brain; balancing autonomic nerve and enhancing sleeping quality; and reducing stress of patient's body and avoiding bedsores/pressure ulcer.

### BRIEF DESCRIPTION OF THE DRAWINGS

FIG. 1 is a block diagram showing the assembly of an adjustment device for a vestibular system of a human body according to a first embedment of the present invention.

FIG. 2 is another block diagram showing the assembly of the adjustment device for the vestibular system of the human body according to the first embedment of the present invention.

FIG. 3 is a top plan view showing the assembly of the adjustment device for the vestibular system of the human body according to the first embedment of the present invention.

FIG. 4 is a side plan view showing the application of the adjustment device for the vestibular system of the human body according to the first embedment of the present invention.

FIG. 5 is another side plan view showing the application of the adjustment device for the vestibular system of the human body according to the first embedment of the present invention.

FIG. 6 is also another side plan view showing the application of the adjustment device for the vestibular system of the human body according to the first embedment of the present invention.

FIG. 7 is still another side plan view showing the application of the adjustment device for the vestibular system of the human body according to the first embedment of the present invention.

FIG. 8 is a perspective view showing the application of an adjustment device for a vestibular system of a human body according to a second embedment of the present invention.

FIG. 9 is a cross sectional view showing the application of a part of the adjustment device for the vestibular system of the human body according to the second embedment of the present invention.

FIG. 10 is a perspective view showing the application of an adjustment device for a vestibular system of a human body according to a third embedment of the present invention.

FIG. 11 is a perspective view showing the application of the adjustment device for the vestibular system of the human body according to the third embedment of the present invention.

### DETAILED DESCRIPTION OF THE PREFERRED EMBODIMENTS

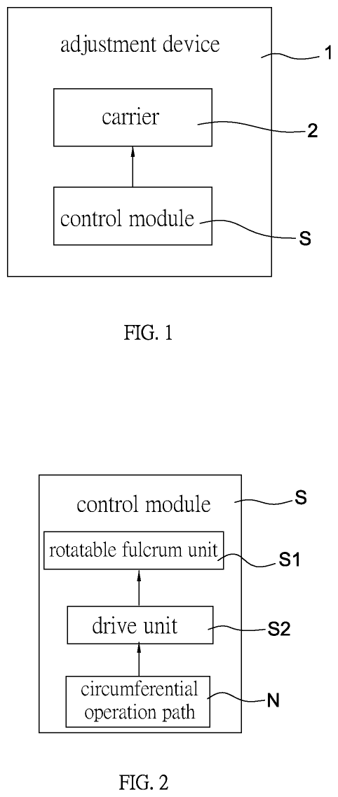

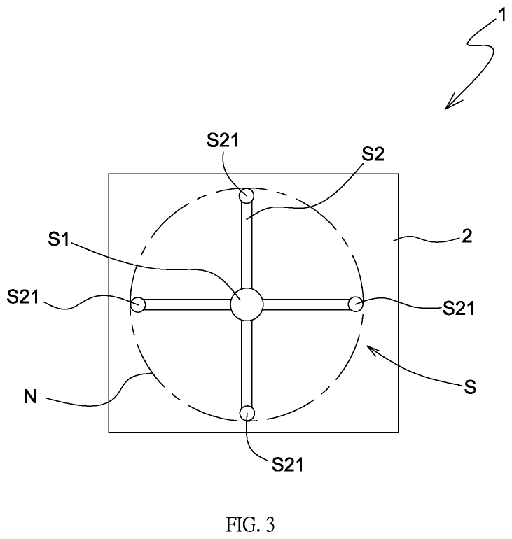

With reference to FIGS. 1-7, an adjustment device 1 for vestibular system of human body according to a first embodiment of the present invention comprises:

a control module S including a rotatable fulcrum unit S1, and the rotatable fulcrum unit S1 having a drive unit S2 rotating along a center of the rotatable fulcrum unit S1, the drive unit S2 having multiple support portions which obliquely rotate upward and downward around a circumferential operation path N, wherein a rotation speed of the drive unit S2 is within 5 rpm to 20 rpm; and

a carrier 2 connected with the multiple support portions of the drive unit S2.

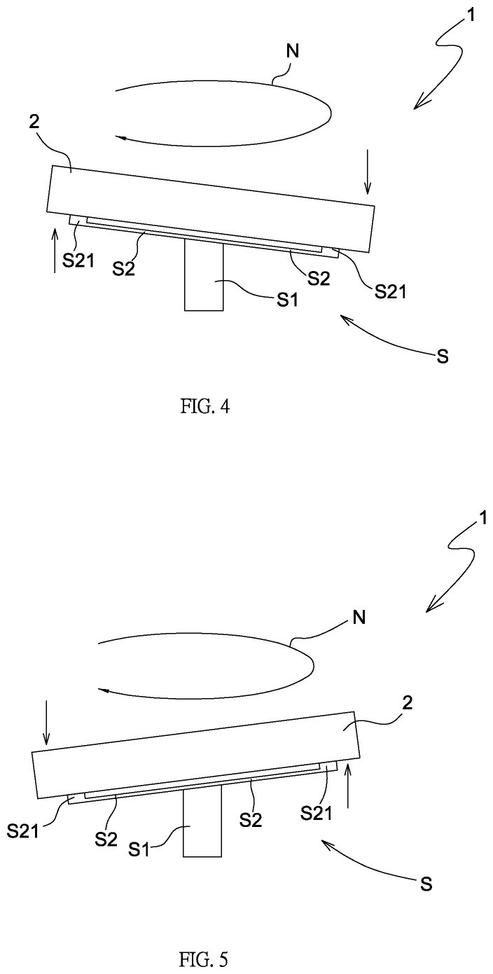

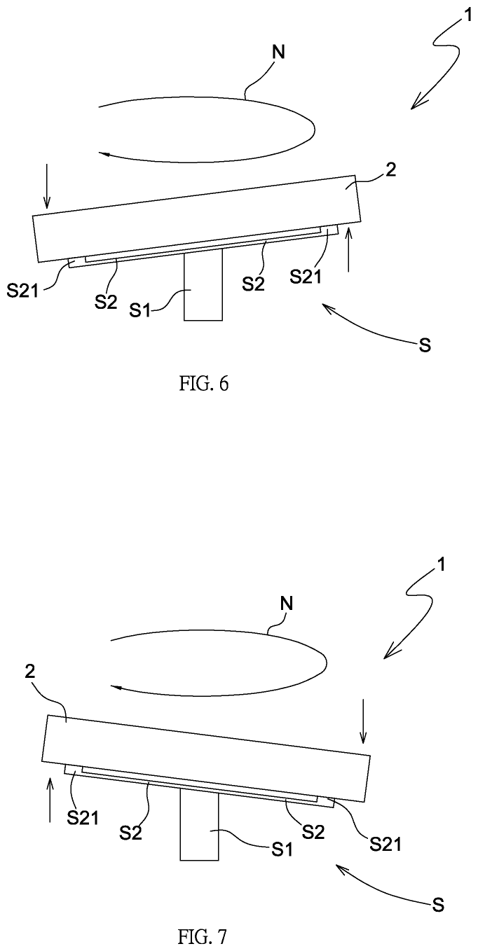

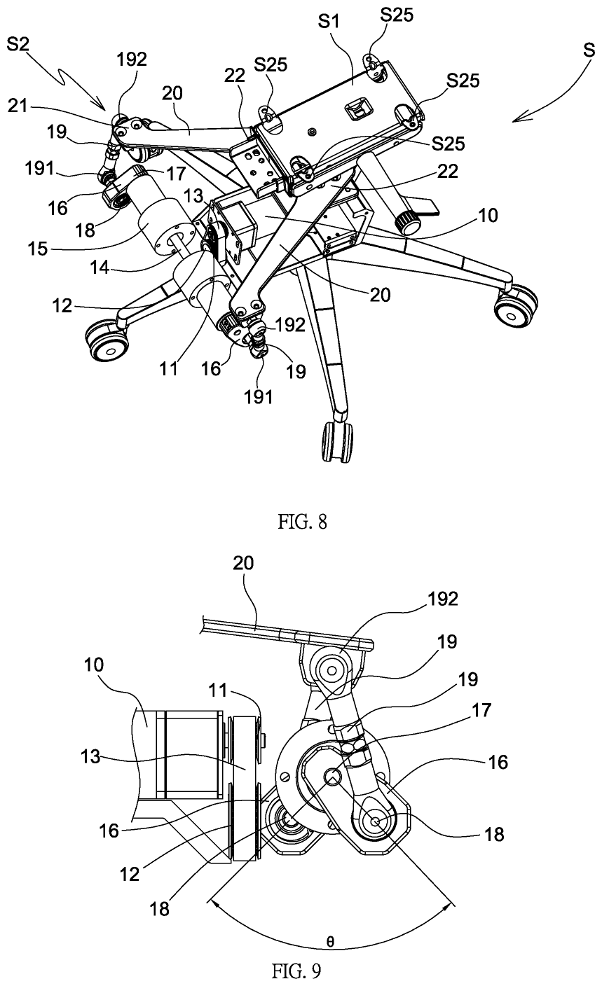

Referring to FIGS. 3-7, the drive unit S2 of the control module S includes four support portions S21 which obliquely rotate upward and downward around the rotatable fulcrum unit S1 in the circumferential operation path N. As shown in FIGS. 4-5, two opposite support portions S21 obliquely rotate upward and downward in the circumferential operation path N. As illustrated in FIGS. 6-7, the other two opposite support portions S21 obliquely rotate upward and downward in the circumferential operation path N.

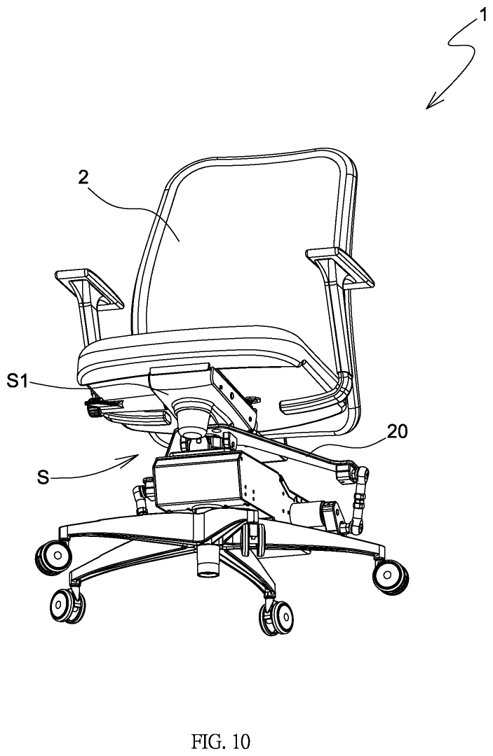

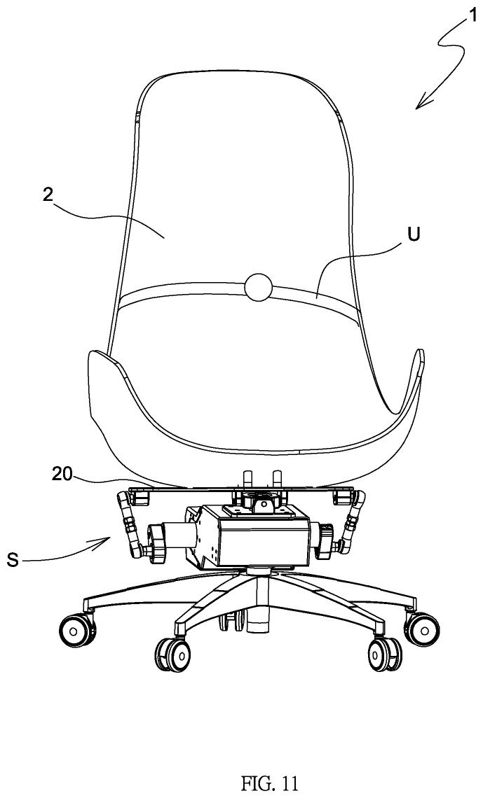

With reference to FIGS. 8-10, an adjustment device 1 for vestibular system of human body according to a second embodiment of the present invention comprises the drive unit S2, and the drive unit S2 includes a motor 10, a drive wheel 11 driven by the motor 10, a deceleration wheel 12 actuated by the drive wheel 11, a belt 13 configured to drive the drive wheel 11 and the deceleration wheel 12, an actuation shaft 14 driven by the deceleration wheel 12, two transmission members 15 arranged on two ends of the actuation shaft 14 respectively so that one of the two transmission members 15 drives an output end 17 of two eccentric shafts 16, and the one output end 17 eccentrically actuates one of two eccentric ends 18. Thereafter, the one eccentric end 18 drives one of two universal input segments 191 of two universal connection rods 19, wherein each universal connection rod 19 has a universal output segment 192 opposite to the universal input segment 191 so that the universal input segment 191 outputs power to the universal output segment 192, and the universal output segment 192 outputs the power to two receiving segments 21 of two guide racks 20 individually, wherein each guide rack 20 includes a sending segment 22 so that the receiving segment 21 outputs the power to the sending segment 22, and the sending segment 22 outputs the power to a rotatable fulcrum unit S1, wherein the rotatable fulcrum unit S1 includes multiple connection portions S25 arranged on four corners thereof individually. As shown in FIG. 9, a deviation angle θ between the two eccentric shafts 16 is 80 degrees to 100 degrees so that each guide rack 20 drives each connection portion S25 of the rotatable fulcrum unit S1 to obliquely rotate upward and downward around the circumferential operation path N.

The carrier 2 is any one of office chair, work chair, children's chair, parent-child chair, massage chair, recliner, infant cradle, baby cradle, hospital bed, home bed, wheelchair, rehabilitation training platform, and equestrian chair.

Referring to FIG. 11, in a third embodiment, the carrier 2 is in connection with a waist protection device U so as to reinforce abdominal muscles and intrinsic muscle of spine and to release back.

Preferably, the carrier 2 is swingable so as to irritate user's feeling and to activate brain.

Accordingly, the adjustment device 1 is applicable for stimulate vestibular system by obliquely rotating the first, second, third, and fourth support portions upward and downward around the circumferential operation path N so as to activate human brain and cerebellum.

The application of the vestibular system of the human body contains:

1. Activating vestibular system of children and improving brain development.

2. Relieving pains of shoulder, neck, and lower back.

3. Stabilizing emotions and easing anxiety.

4. Activating the brain and delaying degradation of the brain.

5. Balancing autonomic nerve and enhancing sleeping quality.

6. Reducing stress of patient's body and avoiding bedsores/pressure ulcer.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. An adjustment device for vestibular system of human body comprising:
a control module including a rotatable fulcrum unit, and the rotatable fulcrum unit having a drive unit rotating along a center of the rotatable fulcrum unit, the drive unit having four support portions which obliquely rotate upward and downward around the rotatable fulcrum unit in a circumferential operation path;
wherein the drive unit includes a motor, a drive wheel driven by the motor, a deceleration wheel actuated by the drive wheel, a belt configured to drive the drive wheel and the deceleration wheel, an actuation shaft driven by the deceleration wheel, two transmission members arranged on two ends of the actuation shaft respectively so that one of the two transmission members drives an output ends of one of two eccentric shafts, and the one output end eccentrically actuates one of two eccentric ends, thereafter the one eccentric end drives one of two universal input segments of two universal connection rods;
wherein each universal connection rod has a universal output segment opposite to the universal input segment so that the universal input segment outputs power to the universal output segment, and the universal output segment is connected with the rotatable fulcrum unit;
wherein a carrier connected with the four support portions of the drive unit.

2. The adjustment device as claimed in claim 1, wherein the universal output segment outputs the power to two receiving segments of two guide racks individually, and each guide rack includes a sending segment so that the receiving segment outputs the power to the sending segment, and the sending segment outputs the power to a rotatable fulcrum unit, wherein the rotatable fulcrum unit includes multiple connection portions arranged on four corners thereof individually, each guide rack drives each connection portion of the rotatable fulcrum unit to obliquely rotate upward and downward around the circumferential operation path, wherein a deviation angle between the two eccentric shafts is 80 degrees to 100 degrees.

3. The adjustment device as claimed in claim 1, wherein the carrier is any one of office chair, work chair, children's chair, parent-child chair, massage chair, recliner, infant cradle, baby cradle, hospital bed, home bed, wheelchair, rehabilitation training platform, and equestrian chair.

4. The adjustment device as claimed in claim 1, wherein the carrier is in connection with a waist protection device.

5. The adjustment device as claimed in claim 1, wherein a rotation speed of the drive unit is within 5 rpm to 20 rpm

6. The adjustment device as claimed in claim 1, wherein application of the vestibular system of the human body contains:
activating vestibular system of children and improving brain development;
relieving pains of shoulder, neck, and lower back;
stabilizing emotions and easing anxiety;
activating the brain and delaying degradation of the brain;
balancing autonomic nerve and enhancing sleeping quality; and
reducing stress of patient's body and avoiding bedsores/pressure ulcer.
